SEQUENCE LISTING (16 sequences)

SEQ ID NO: 1 - protein, 9 aa, Homo sapiens
KIFGSLAFL

SEQ ID NO: 2 - protein, 9 aa, Homo sapiens
RLLQETELV

SEQ ID NO: 3 - protein, 15 aa, Homo sapiens
HLDMLRHLYQGCQVV

SEQ ID NO: 4 - protein, 17 aa, Homo sapiens
RLRIVRGTQLFEDNYAL

SEQ ID NO: 5 - protein, 18 aa, Homo sapiens
TQRCEKCSKPCARVCYGL

SEQ ID NO: 6 - protein, 15 aa, Homo sapiens
GVGSPYVSRLLGICL

SEQ ID NO: 7 - protein, 15 aa, Homo sapiens
GVGSPYVSRLLGICL

SEQ ID NO: 8 - protein, 15 aa, Homo sapiens
TLERPKTLSPGKNGV

SEQ ID NO: 9 - protein, 15 aa, Homo sapiens
SWPPHMHNFSVFSNL

SEQ ID NO: 10 - protein, 15 aa, Homo sapiens
TTIGGRSLYNRGFSL

SEQ ID NO: 11 - protein, 15 aa, Homo sapiens
AGRIYISANRQLCYH

SEQ ID NO: 12 - protein, 15 aa, Homo sapiens
VADFGVADLLPPDDK

SEQ ID NO: 13 - protein, 15 aa, Homo sapiens
QLLYSEAKTPIKWMA

SEQ ID NO: 14 - protein, 15 aa, Homo sapiens
VPDLLEKGERLAQPQ

SEQ ID NO: 15 - protein, 15 aa, Homo sapiens
GCLASESSEGHVTGS

SEQ ID NO: 16 - protein, 15 aa, Homo sapiens
EAELQEKVSMCRSRS

(19) United States
(12) Patent Application Publication
(10) Pub. No.: US 2024/0299544 A1
(43) Pub. Date: Sep. 12, 2024

(54) CANCER THERAPIES COMPRISING PEPTIDE LOADED CXCR3- AND CCR5-INDUCING DENDRITIC CELLS AND CHEMOKINE MODULATORY AGENTS

(71) Applicants: Health Research, Inc., Buffalo, NY (US); H. Lee Moffitt Cancer Center and Research Institute, Inc., Tampa, FL (US)

(72) Inventors: Pawel KALINSKI, East Aurora, NY (US); Brian CZERNIECKI, Tampa, FL (US)

(21) Appl. No.: 18/569,178

(22) PCT Filed: Jun. 16, 2022
(86) PCT No.: PCT/US2022/033885

§ 371 Date: Dec. 11, 2023

Related U.S. Application Data
(60) Provisional application No. 63/211,150, filed on Jun. 16, 2021.

(51) Int. Cl. A61K 39/00; A61K 31/415; A61K 31/713; C07K 16/28; C12N 5/0784

(52) U.S. Cl. CPC A61K 39/4615; A61K 31/415; A61K 31/713; A61K 39/4622; A61K 39/464406; C07K 16/2827; C12N 5/0639; A61K 2039/505; C12N 2501/056; C12N 2501/2301; C12N 2501/24; C12N 2501/25

(57) ABSTRACT

Provided are compositions and methods for prophylaxis or therapy of cancer. The compositions comprise α-type-1 dendritic cells that have been treated with intact proteins that comprise cancer antigens, or peptides that comprise cancer antigens, or combinations thereof. The approaches can also include adding a chemokine-modulating regimen.

### CROSS REFERENCE TO RELATED APPLICATION

This application claims priority to U.S. provisional application No. 63/211,150, filed Jun. 16, 2021, the entire disclosure of which is incorporated herein by reference.

### SEQUENCE LISTING

The instant application contains a Sequence Listing which has been submitted in ASCII format and is hereby incorporated by reference in its entirety. Said ASCII copy, created on Jun. 65, 2022, is named “003551_01051_ST25.txt”, and is 3,498 bytes in size.

### BACKGROUND

Breast Cancer is the second leading cause of brain metastasis following lung cancer. HER3, overexpressed in BMBC is a resistance factor to HER2-targeting therapies and a driver of CNS metastasis. Progression of HER2⁺ BC and triple-negative BC (TNBC) is associated with loss of anti-HER2- and anti-HER3 immunity. About 15-30% of patients with Stage IV breast cancer develop brain metastasis (BM). HER2 and triple negative breast cancer (TNBC) both have a predilection to metastasize to the brain and CNS. HER2 breast cancers have improved systemic control with HER2 targeted therapies but these patients have increased risk of developing BM. Overall survival of HER2 patients developing brain metastasis is 16.5 months while TNBC is 4.9 months so these patients are in need of new therapies to reduce mortality. The present disclosure is related to this and other needs pertaining to cancer patients.

### BRIEF DESCRIPTION OF THE DRAWINGS

FIG. 1. CKM selectively induces Th1/CTL attractants, in ex-vivo-cultured human brain-metastatic TNBC explant. Resected brain-metastatic TNBC tissues were cultured at the interphase of medium and air in our ex vivo tumor explant model 6-10 in the absence or presence of CKM (rintatolimod+IFNα+celecoxib) for 24 hrs. The intratumoral expression levels of CXCL10, CCL5, and CCL22 (relative to HPRT1) were measured by quantitative RT-PCT (Taqman).

FIG. 2. Expected changes in the tumor microenvironment induced by the combination of αDC1 vaccine (or by adoptively transferred ex vivo-αDC1-sensitized T cells), CKM and PD-1 blockade. At baseline, the tumor has an immunosuppressive microenvironment with MDSC and Treg and immunosuppressive chemokines (CXCL12, CCL2 and CCL22), but treatment with αDC1 vaccine and CKM induces tumor infiltration with CTL, Th1 cells and NK cells by increasing the production of CTL attractants (CXCL9, CXCL10, CXCL11 and CCL5). This creates an immune enriched microenvironment. It is expected that combining CKM and the described αDC1 vaccine will increase the efficacy of PD-1 checkpoint inhibitors.

FIG. 3. HER3 protein (5 μg/mL)-loaded αDC1s induce higher numbers of HER3-specific CD4+ T cells population, compared with αDC1s loaded with multiple HER3 peptides (5 μg/mL each) in in vitro sensitization cultures involving autologous CD4 T cells and DCs. The numbers of the resulting IFNγ-producing cells in the differentially-expanded populations were determined by ELISPOT assay using iDC loaded with HER3 protein (2 μg/mL) as target cell. Target cells were cocultured with CD4+ T cell at the ratio of 1 to 10 for 36 hr before the spots were developed and counted.

FIG. 4. HER3 protein (5 μg/mL) loaded αDC1s induce HER3-specific responses within CD8 population. IFNγ production was determined by ELISPOT assay. Briefly, iDCs were loaded with HER3 protein (2 μg/mL) and used as target cell. Target cell was cocultured with CD8+ T cell (at the ratio of 1 to 10) for 36 hr before the spots were developed and counted.

FIG. 5. HER3 protein (5 μg/mL) loaded αDC1s induce similar responses against epitopes from previously identified (Czerniecki) HER3 peptides within CD4 population as compared with HER3 peptides (5 μg/mL each) loaded αDC1s. The numbers of the resulting IFNγ-producing T cells reactive to each of the individually-loaded Her3 peptides (2 μg/mL) were determined by ELISPOT assay. Briefly, iDC was loaded with individual HER3 peptide (2 μg/mL) and used as target cell. Target cell was cocultured with the differentially expanded CD4+ T cell at the ratio of 1 to 10 for 36 hr before the spots were developed and counted.

### BRIEF SUMMARY

The present disclosure provides compositions and methods that are used for prophylaxis or therapy of cancer. The compositions comprise, in part, a specialized type of protein or peptide loaded dendritic cells (DCs), which induce high levels of two chemokine receptors, CCR5 and CXCR3 on the activated T cells. The DCs are used as vaccines or ex vivo inducers of T cells for adoptive cancer therapies, either alone or in conjunction with a tumor-selective Chemokine Modulatory regimen (CKM), which induces specific chemokines which bind CCR5 and CXCR3, such as CCL5 and CXCL9, CXCL10 and CXCL11, in tumor tissues. In certain embodiments the compositions and methods are used to promote the accumulation of CCR5/CXCR3-expressing tumor-specific T cells in the tumor tissues and thus potentiate the therapeutic effectiveness of immune checkpoint blockade.

In an embodiment, the disclosure provides a method for treating cancer in an individual in need thereof. In general, the cancer will be comprised by at least one tumor. In embodiments, the tumor may have metastasized. In a non-limiting embodiment, the tumor is a breast cancer tumor. In an embodiment, the breast cancer has metastasized to the brain.

Methods provides by the disclosure generally comprise administering to the individual: a) a combination of autologous dendritic cells loaded with at least one MHC Class II- and/or at least one MHC class I-restricted Her2 and Her3 peptide, wherein the peptide may be displayed by MHC-I or MHC-II by loading the DCs with peptide, or via internal dendritic cell processing of an intact protein supplied to the dendritic cells; and optionally b) a combination of agents, said combination of agents having a tumor selective chemokine-modulating (CKM) effect.

In embodiments, the dendritic cells are alpha-type-1 dendritic cells (αDC1s). In embodiments, the DCs are optionally matured in the presence of IFNα and IFNγ, and optionally in the presence of one or a combination of IL-1, TNF, and poly-I:C.

In embodiments, the dendritic cells are matured in the presence of the combination of IFNα and IFNγ, and in the presence of at least two of IL-1, TNF, and poly-I:C, or in the presence of all of IFNα, IFNγ, IL-1, TNF, and poly-I:C. The maturation can be performed in the presence of the described agents concurrently, or consecutively.

In embodiments, the described DCs display peptides that are produced by internal processing of a polypeptide. The polypeptide may be an intact polypeptide that comprises MHC Class I epitopes, MHC Class II epitopes, or a combination thereof. The polypeptide may be supplied to the dendritic cells in vitro to produce peptide loaded DCs. In a non-limiting embodiment, the DCs are supplied in vitro with a Her3 protein to thereby provide Her3 protein-loaded DCs. In embodiments, use of the described DCs includes using DCs that have been supplied a polypeptide that comprises peptide segments, instead of providing the DCs with peptides. In an embodiment, the DCs that have been provided with a polypeptide produce a more effective anti-cancer result relative to DCs that are loaded with peptides only. In embodiments, an improved CD4+ T cell response is generated. In embodiments, an improved CD8+ T cell response is generated.

In embodiments, the described DC's are loaded with at least one MHC Class II- and/or at least one MHC class I-restricted Her2 and Her3 peptides. Suitable combinations of peptides are selected from the following:

| Peptide | SEQ ID NO |
|---|---|
| HER2 MHC I - P369-377 | SEQ ID NO: 1 |
| KIFGSLAFL | |
| SHER2 MHC I - P689-697 | SEQ ID NO: 2 |
| RLLQETELV | |
| HER2 MHC II - P42-56 | SEQ ID NO: 3 |
| HLDMLRHLYQGCQVV | |
| HER2 MHC II - P98-114 | SEQ ID NO: 4 |
| RLRIVRGTQLFEDNYAL | |
| HER2 MHC II - P328-345 | SEQ ID NO: 5 |
| TQRCEKCSKPCARVCYGL | |
| HER2 MHC II - P776-790 | SEQ ID NO: 6 |
| HER2 MHC II - P927-941c | SEQ ID NO: 7 |
| HER2 MHC II - P1166-1180 | SEQ ID NO: 8 |
| TLERPKTLSPGKNGV | |
| HER3 MHC II ECD - P81 | SEQ ID NO: 9 |
| (aa 401-415) | |
| SWPPHMHNFSVFSNL | |
| HER3 MHC II ECD - P84 | SEQ ID NO: 10 |
| (aa 416-430) | |
| TTIGGRSLYNRGFSL | |
| HER3 MHC II ECD - P91 | SEQ ID NO: 11 |
| (aa 451-465) | |
| AGRIYISANRQLCYH | |
| HER3 MHC II ICD - P38 | SEQ ID NO: 12 |
| (aa 850-864) | |
| VADFGVADLLPPDDK | |
| HER3 MHC II ICD - P41 | SEQ ID NO: 13 |
| (aa 865-879) | |
| QLLYSEAKTPIKWMA | |
| HER3 MHC II ICD - P52 | SEQ ID NO: 14 |
| (aa 920-934) | |
| VPDLLEKGERLAQPQ | |
| HER3 MHC II ICD - P86 | SEQ ID NO: 15 |
| (aa 1090-1114) | |
| GCLASESSEGHVTGS | |
| HER3 MHC II ICD - P89 | SEQ ID NO: 16 |
| (aa 1115-1129) | |
| EAELQEKVSMCRSRS | |

In one non-limiting embodiment, a Class II peptide is selected from: HER-2/neu peptides:

| | p42-56 |
|---|---|
| (SEQ ID NO: 3) | |
| | (HLDMLRHLYQGCQVV), |
| | p98-114 |
| (SEQ ID NO: 4) | |
| | (RLRIVRGTQLFEDNYAL), |
| | p328-345 |
| (SEQ ID NO: 5) | |
| | (TQRCEKCSKPCARVCYGL), |
| | p776-790 |
| (SEQ ID NO: 6) | |
| | (GVGSPYVSRLLGICL), |
| | p927-941 |
| (SEQ ID NO: 7) | |
| | (PAREIPDLLEKGERL), |
| | and |
| | p1166-1180 |
| (SEQ ID NO: 8) | |
| | (TLERPKTLSPGKNGV); |

HER-3 extracellular domain (ECD) peptides:

| | P81 |
|---|---|
| (SEQ ID NO: 9) | |
| | (aa 401-415 SWPPHMHNFSVFSNL), |
| | P84 |
| (SEQ ID NO: 10) | |
| | (aa 416-430 TTIGGRSLYNRGFSL), |
| | and |
| | P91 |
| (SEQ ID NO: 11) | |
| | (aa 451-465 AGRIYISANRQLCYH), |
| | and |

HER-3 intracellular domain (ICD) peptides:

| | P38 |
|---|---|
| (SEQ ID NO: 12) | |
| | (aa 850-864 VADFGVADLLPPDDK), |
| | P41 |
| (SEQ ID NO: 13) | |
| | (aa 865-879 QLLYSEAKTPIKWMA), |
| | P52 |
| (SEQ ID NO: 14) | |
| | (aa 920-934 VPDLLEKGERLAQPQ), |
| | P86 |
| (SEQ ID NO: 15) | |
| | (aa 1090-1114 GCLASESSEGHVTGS), |
| | and |
| | P89 |
| (SEQ ID NO: 16) | |
| | (aa 1115-1129 EAELQEKVSMCRSRS); |

and a Class II peptide is selected from:

HER-2/neu peptide P369-377 (KIFGSLAFL) (SEQ ID NO:1) and P689-697 (RLLQETELV) (SEQ ID NO:2).

In certain approaches, at least one polypeptide, or at least two of the peptides, or a combination of a polypeptide and at least two peptides, are loaded onto the autologous dendritic cells. In embodiments, the combination of at least two peptides comprises at least one Class II peptide, and optionally comprises at least one Class I peptide. In one embodiment, the combination of at least two peptides comprises a combination of at least one Class II peptide and at least one Class I peptide.

In an embodiment, the disclosure comprises administering a combination the described DCs and a combination of agents having the tumor selective CKM effect. In non-limiting embodiments, the combination of agents having the selective CKM effect comprises a combination of at least two of:
i) a COX-2 inhibitor that is optionally Celecoxib.
ii) an interferon that is optionally human recombinant Interferon Alpha-2b, and
iii) a poly IC analog (double-stranded RNA) that is optionally Rintatolimod.

In a non-limiting embodiment, the COX-2 inhibitor is Celecoxib, the interferon is a human recombinant Interferon Alpha-2b, and the poly IC analog is Rintatolimod.

In certain embodiments, the DCs comprise autologous DCs that are matured in the presence of at least one of the described peptides, and in the presence of cytokines, the cytokines comprising a combination of at least two of GM-CSF, IFNα, IFNγ, IL1β, TNFα and poly-I:C.

In a non-limiting embodiment, approximately 10×10⁶ of peptide loaded αDCs cells are administered to the individual. In an embodiment, the peptide loaded αDCs cryopreserved prior being administered to the individual.

In an embodiment, the autologous «DCs are provided as a combination therapy with at least one of: an adjuvant, a cytokine, an inhibitor of at least one checkpoint molecule, or a suppressive factor. In an embodiment, the combination therapy comprises the inhibitor of the checkpoint molecule. In an embodiment, the autologous «DCs and the combination of CKM agents sensitizes the individual to the inhibitor of the checkpoint molecule. In an embodiment, the checkpoint molecule inhibits at least one of PD1, PD-L1 or PD-L2, or CTLA4. In a non-limiting embodiment, the inhibitor of the checkpoint molecule comprises Pembrolizumab.

The disclosure also provides a population of isolated αDCs having loaded thereon one or more of the described peptides.

In one embodiment, the population of isolated αDCs are derived from a monocyte culture that is exposed to one or more the described peptides and a combination of the described cytokines.

In an embodiment, a pharmaceutical composition comprising a population of isolated αDCs is provided.

In one embodiment, the disclosure provides for electing an individual for treatment of a cancer, the cancer comprising at least one tumor, isolating a liquid biological sample comprising at least peripheral blood mononuclear cells (PBMCs) from the individual, separating monocytes from the PBMCs, culturing the monocytes in the presence of at least one the described peptides, an intact protein, or a combination of peptides and an intact protein, and optionally a combination of cytokines comprising at least two of GM-CSF, IFNα, IFNγ, IL1β, TNFα and poly-I:C. This provides autologous αDCs comprising MHC II or MHC I molecules, or a combination thereof, loaded with at least one of the described peptides.

In an embodiment, the αDCs administered to the individual are alpha-type-1-polarized DCs. In an embodiment, the described method further comprises administering to the individual a combination of agents selected from the group of agents consisting of:
i) a COX-2 inhibitor that is optionally Celecoxib,
ii) an interferon that is optionally human recombinant Interferon Alpha-2b, and
iii) a poly IC analog that is Rintatolimod.

In an embodiment, the method further comprises administering to the individual least one of: an adjuvant, a cytokine, an inhibitor of at least one checkpoint molecule, or a suppressive factor. In an embodiment, the combination therapy comprises the inhibitor of the checkpoint molecule. In an embodiment, the autologous αDCs and the combination of CKM agents sensitize the individual to the inhibitor of the checkpoint molecule. In embodiments, the inhibitor of the checkpoint molecule inhibits at least one of PD1, PD-L1 or PD-L2, or CTLA4. In an embodiment, the tumor was resistant to the inhibitor of the checkpoint molecule prior to the administration of the autologous αDCs cells and the combination of CKM agents, and the administration of the autologous αDCs cells and the combination of CKM agents sensitizes the individual to the inhibitor of the checkpoint molecule.

### DETAILED DESCRIPTION

The present disclosure provides compositions and methods that are used for prophylaxis and/or therapy of cancer. The compositions comprise α-type-1 dendritic cells (“αDC1s) that are prepared using compositions and methods as further described below.

In certain approaches the described compositions and methods are expected exhibit a synergistic effect. In a non-limiting embodiment, a synergistic effect comprises a therapeutic synergy that includes promoting the induction and/or expansion of Her2/3-specific T cells and their enhanced accumulation in tumor tissues, thus enhancing local immune control and sensitizing cancer cells to the therapeutic effects of PD-1 inhibition. Thus, in embodiments, the compositions and methods improve a cancer patient's response to immune checkpoint inhibitors, including but not necessarily limited to PD-1 blockade. The PD1 inhibition may comprise use of an agent that binds to the PD-1, its ligand (PD-L1), or a combination of such agents. In general, PD-1 blockade is achieved using monoclonal antibodies, non-limiting examples of which bind to PD-1 and include Pembrolizumab, sold under the brand name KEYTRUDA, Nivolumab, sold under the brand name OPDIVO, and Cemiplimab sold under the brand name LIBTAYO. Each of these monoclonal antibodies are FDA approved for certain indications, but the disclosure includes any other agents that can be used in PD-1 blockade by binding to PD-1, such as JTX-4014, Spartalizumab, Camrelizumab, Sintilimab, Tislelizumab, Toripalimab, Dostarlima, AMP-224, and AMP-514. In embodiments, the PD-1 blockade is achieved using a PDL1 inhibitor, examples of which include but are not limited to Atezolizumab sold under the brand name TECENTRIQ, Avelumab sold under the brand name BAVENCIO, and Durvalumab sold under the brand name IMFINZI. Other biologics and chemotherapeutic agents may be combined with the DC vaccines of the present disclosure. For example, use of trastuzumab and/or pertuzumab is included in the disclosure.

In embodiments, the checkpoint inhibitor inhibits the interaction between T cell expressed PD-1 and either PD-L1 or PD-L2 (expressed in tumor tissues and by antigen-presenting cells) or the interaction between T cell-expressed CTLA4 and either CD8 or CD86 expressed in the tumor tissues and by antigen-presenting cells Other checkpoint inhibitors block the interactions between additional T cell-expressed checkpoints, including Lag-3 and Tim-3 and their ligands expressed in the tumor tissues and by antigen-presenting cells.

In embodiments, the PD1 inhibitor comprises pembrolizumab, nivolumab, avelumab, and cemiplimab, atezolizumab avelumab, durvalumab, sintilimab, tislelizumab, toripalimab, dostarlimab, INCMGA00012, AMP-224, AMP-514, NKJ035, CK-301, AUNP12, CA-170 or BMS-986189. CTLA4 inhibitor comprises ipilimumab or tremelimumab.

The disclosure includes certain improvements relative to previously available compositions and methods. The advantages include but are not limited to use of autologous αDCs which are prepared using novel combinations of agents, non-limiting examples of which are described below. Additionally, the present disclosure provides a novel tumor-selective Chemokine Modulatory regimen (CKM), as further described herein. In embodiments, the CKM approach includes preparation of an αDC1 vaccine using autologous cell obtained and exposed to certain agents as further described herein. In an embedment, the CKM approach uses autologous peripheral blood monocytes grown in GM-CSF and IL-4, matured using a combination of TNF-α, IL-1-β, poly-I:C, interferon-α (IF-α) and interferon-γ (IFN-γ). The autologous cells are loaded with several MHC class II binding peptides and MHC class I binding peptides, non-limiting examples of which are described below. The CKM comprises use of a double stranded RNA agent, which generally comprises inosinic and cytidylic acid residues and as such contains poly I:poly C or poly I:C. A non-limiting example of a suitable RNA agent is Rintatolimod, sold under the tradename AMPLIGEN. The CKM approach further comprises use of IF-α, and a COX-2 inhibitor, a non-limiting example of which comprises Celecoxib, sold under the brand name CELEBREX.

In embodiments, the present disclosure differs from previous approaches in that autologous αDC1s are loaded with MHC class I and MHC class II-restricted antigenic peptides corresponding to Her2 and Her3 (which are known in the art as breast cancer-relevant antigens), rather than tumor-blood vessel-targeting antigens. Thus, in embodiments, the compositions and methods of this disclosure may be free of blood-vessel tumor antigens. In embodiments, the antigens are not ovarian cancer (OvCa) antigens. In embodiments, the antigens are not colorectal cancer (CRC) antigens. In embodiments, the antigens are not melanoma antigens.

In one aspect the disclosure use of the described peptide loaded αDC1 to induce (either in vivo, when used as vaccines, or ex vivo when used to induce tumor specific cells for adoptive T cell therapies) high numbers of tumor-specific T cell which express high levels of CR5 and CXCR3 and the CKM to induce matching intra-tumoral production of the chemokine ligands for CCR5 and CXCR3, such as CCL5, CXCL9, CXCL10 and CXCL11. In embodiments, this approach preferentially activates cancer tissues, rather than surrounding tissues, resulting in preferential homing of αDC1-induced CTLs, as well as and other type-I immune cells to tumors, examples of which include but are not necessarily limited to T-bet” IFN-γ-producing group 1 ILCs (e.g., ILC1 and natural killer cells), CD8⁺ cytotoxic T cells (CDLs), and CD4⁺ T_{H}1 cells. Thus, in various embodiments, the disclosure provides for improving a cell mediated immune response directed against cancer cells, which may be comprised by one or more tumors, and which may exert such an immune response in a localized tumor environment. In embodiments, administration of a described cellular composition to an individual in need thereof induces Th1 cells and cytotoxic T cells (CTLs) which express the chemokine receptors CCR5 and CXCR3.

In embodiments, a therapeutically effective amount of a composition of this disclosure is administered. In embodiments, a composition comprising autologous αDCs modified according to this disclosure is administered in a therapeutically effective amount, e.g., a dosage. A precise dosage can be selected by the individual physician in view of the patient to be treated. Dosage and administration can be adjusted to provide sufficient amounts of the αDCs to achieve and maintain the desired effect. Additional factors which may be taken into account include the stage and type of cancer, the age, weight and gender of the patient, desired duration of treatment, method of administration, time and frequency of administration, drug combination(s), reaction sensitivities, and tolerance/response to therapy. A therapeutically effective amount is an amount that reduces one or more signs or symptoms of a disease, and/or reduces the severity of the disease. A therapeutically effective amount may also inhibit or prevent the onset of a disease, or a disease relapse. In embodiments, a therapeutically effective amount is an amount that reduces or eliminates cancer cells from an individual. In embodiments, a therapeutically effective dose inhibits growth of cancer cells, such as cancer cells in a tumor. In embodiments, a therapeutically effective dose inhibits formation of a primary tumor, and/or inhibits metastasis from a tumor.

The type of cancer treated according to this disclosure is not particularly limited, other than in connection with the particular peptides used to load the αDCs, and a tumor formation. In embodiments, cancer cells that are affected according to this disclosure include but are not necessarily limited to breast cancer, prostate cancer, pancreatic cancer, lung cancer, liver cancer, ovarian cancer, cervical cancer, colon cancer, esophageal cancer, stomach cancer, bladder cancer, brain cancer, testicular cancer, head and neck cancer, melanoma, skin cancer, any sarcoma, including but not limited to fibrosarcoma, angiosarcoma, adenocarcinoma, and rhabdomyosarcoma. In embodiments, the individual is in need of treatment for breast cancer. In embodiments, the individual is need of treatment for metastatic breast cancer. In one embodiment, the individual is need of treatment for brain-metastatic breast cancer. In embodiments, the individual has a breast cancer and has an absent or deficient CD4+Th1 cell response, and therefore may have a poor prognosis which may be addressed using the described anti-HER2 DC vaccines. In embodiments, the individual has HER2 positive or triple negative breast cancer (TNBC). In embodiments, a composition comprising the described αDCs is administered to an individual who previously had cancer, or is at risk for developing cancer, and thus prophylactic approaches are included by this disclosure.

In embodiments, the disclosure comprises selecting an individual who has been diagnosed with cancer, and administering a described composition to the individual. The method may further comprise testing the individual to determine the efficacy of the described therapy, e.g., monitoring the status of the cancer in the individual over a period of time subsequent to, or during a dosing regimen. The compositions comprising the described cells may be specifically targeted to cancer cells.

In embodiments, the described dendritic cells (or αDC1-induced T cells) are administered to an individual using any suitable route. Thus, the administration may comprise parenteral, subcutaneous, intraperitoneal, intracranial, and intra-tumoral administrations. Parenteral infusions include intramuscular, intravenous, intraarterial, intraperitoneal, and subcutaneous administration. In embodiments, the administration is an intralymphatic, intranodal, or intradermal routes of αDC1 administration.

In embodiments, the described cells can be provided as a pharmaceutical composition. In embodiments, pharmaceutical compositions comprise isolated «DCs as described herein together with any suitable pharmaceutically acceptable carriers, excipients and/or stabilizers. Examples of pharmaceutically acceptable carriers, excipients and stabilizer can be found in Remington: The Science and Practice of Pharmacy (2005) 21st Edition, Philadelphia, PA. Lippincott Williams & Wilkins, the disclosure of which is incorporated herein by reference.

In connection with certain embodiments as described above and elsewhere in this disclosure, in a non-limiting approach, the described compositions and methods are used for treating breast cancer. In this regard, breast cancer is the second leading cause of brain metastasis following lung cancer. HER3, overexpressed in BMBC is a resistance factor to HER2-targeting therapies and a driver of CNS metastasis. Progression of HER2+BC and triple-negative BC (TNBC) is associated with loss of anti-HER2- and anti-HER3 immunity. About 15-30% of patients with Stage IV breast cancer develop brain metastasis (BM) HER2 and triple negative breast cancer (TNBC) both have a predilection to metastasize to the brain and CNS. HER2 breast cancers have improved systemic control with HER2 targeted therapies but these patients have increased risk of developing BM. Overall survival of HER2 patients developing brain metastasis is 16.5 months while TNBC is 4.9 months so these patients are in need of new therapies to reduce mortality, and as such these patients are particularly pertinent to the present disclosure.

There is evidence that disseminated cancer cells (DCC) and circulating tumor cells (CTC) may lead to dissemination of breast cancer cells to the brain. The EGFR pathway is involved in signaling in breast BM in TNBC and HER2 breast cancers. These same pathways are active in glioblastoma. Interestingly data suggests HER3 is specifically increased in BM. The receptor binds Heregulin that is expressed abundantly in the brain tissue. HER3 is binding partner with HER2 and the pair is the most oncogenic signaling pair. HER3 can also be overexpressed in TNBC and its expression is associated with poor prognosis. Recent data suggests targeting HER3 may be essential to achieve responses using HER2 targeted agents in breast BM. HER3 may be critically involved in the seed soil of breast BM. Hence, in embodiments, the present disclosure relates to overexpression and targeting of this protein with the described DC vaccines.

Alpha-type-1-polarized dendritic cells (αDC1s) are particularly effective in the induction of effector functions in CTL precursors, and in inducing high levels of peripheral tissue-homing receptors CCR5 and CXCR3.

In invasive breast cancer (IBC), cytotoxic T cell (CTL) infiltration of the tumor predicts both, improved response to therapy and overall survival. This is true in multiple other tumor types. It also facilitates responsiveness of multiple cancer types to checkpoint blockers, the new class of highly potent immunotherapeutic. In contrast, intratumoral prevalence of regulatory T cells (Tregs) is associated with poor tumor response and impaired survival in breast cancer and other tumors. These findings highlight the potential for modulating TIL densities in the management of breast cancer. Accordingly, enhanced infiltration of CD8⁺ T cells following modulation of TME can improve spontaneous control of tumor growth and the effectiveness of PD1 and CTLA4 blockade in multiple mouse models.

Intratumoral T cell (infiltration predicts clinical outcomes in patients with breast as well as other cancers and their responsiveness to PD1/PDL1 blockade. Density of tumor-infiltrating CTLs in CRC patients are strong predictors of survival, independent of the disease stage. High levels of effector- and effector/memory CD8⁺ T cells (CTLs) in CRC predict improved overall survival (OS) and the effectiveness of both chemotherapy and therapeutic blockade of programmed death-1 (PD-1) pathway. In contrast to the desirable CTLs and Th1 cells, intratumoral regulatory T cells (Tregs) predict poor outcomes, indicating therapeutic potential for selectively enhancing intratumoral CTL densities, relative to Tregs. Accordingly, enhanced infiltration of CTLs triggered by local (intratumoral) injection of a STING activator (cGAMP) can suppress tumor growth.

Heterogeneity of CTL infiltration involves not only the differences between cancers and patients, but also individual tumor lesions of the same patient. Such heterogeneity and the ability of cancers to adapt to immune pressure during tumor progression help explain primary and secondary unresponsiveness to PD-1 blockers and other immunotherapies and highlight the need for new means to induce homogenous CTL infiltration of all lesions within a patient, to which the present disclosure is pertinent.

The chemokine receptors CXCR3 and CCR5 are selectively expressed on immune effector cells, such as CTLs and Th1 cells, as well as activated NK cells. High tumor production of CCL5/RANTES (ligand for CCR5) and CXCL9/MIG, CXCL10/IP10, and CXCL11/ITAC (three known ligands for CXCR3) is associated with high CTL infiltration in multiple cancer types, while Tregs are selectively attracted by CCL22/MDC. Tight correlations between TME production of CCL5, CXCL9, CXCL 10 and local infiltration with CD8⁺GrB⁺ CTLs, and correlation of CCL22 with Treg markers have been shown. Most of the CCL5 and all of the CXCL10 are produced by non-CTLs, indicating the causative role of these two chemokines in CTL attraction.

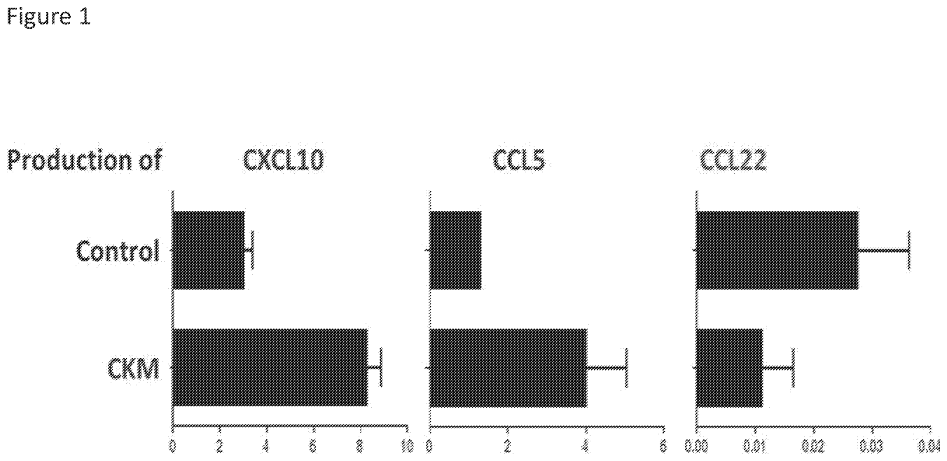

The instant disclosure demonstrates the ability of the presently described approach, e.g., use of CKM, to reprogram brain-metastatic breast cancer tissues. Using an established ex vivo tumor explant model, the disclosure demonstrates that the TMEs of non-CNS breast cancer and (potentially more suppressive) BMBC can be reprogramed to a similar extent by CKM ex vivo to selectively induce Th1/CTL attractants CXCL 10 and CCL5 without enhancing the intratumoral production of Treg-attractant CCL22. (See FIG. 1).

The ex vivo tumor model is described in, for example, Muthuswamy, R., et al. J Immunother Cancer 3, 6 (2015); Obermajer, N., et al., Nat Protoc 13, 335-357 (2018); Theodoraki, M. et al., Cancer Res 78, 4292-4302 (2018); Aversa, C., et al., Breast 23, 623-628 (2014); and Hayashi, N., et al. Breast Cancer Res Treat 149, 277-284 (2015), from which the descriptions of ex vivo tumor models in incorporated herein by reference.

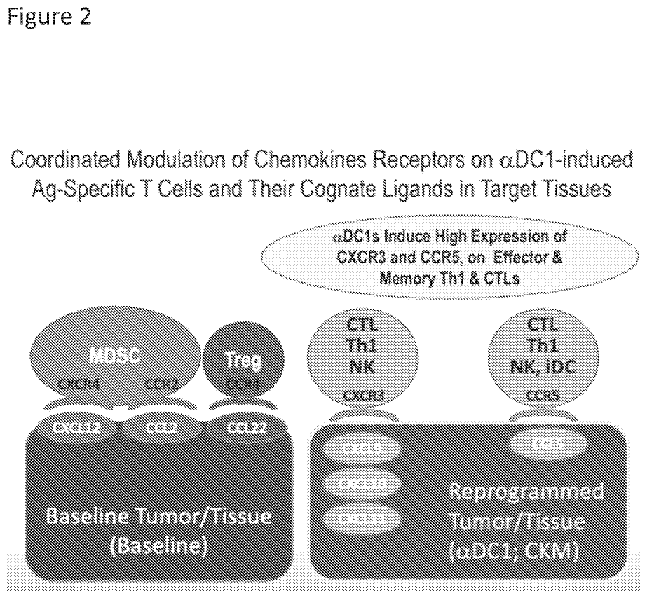

Based in part on the data and description of this disclosure, the invention includes, as described above, treatment comprising combining αDC1 vaccine-loaded with BMBC-relevant peptide antigens and CKM, to sensitize breast cancer patients with CNS metastasis to PD1 inhibition. Further, we have demonstrated that using CKM on days −11, −10, −9 and −4, −3, −2+Pembrolizumab every 3 weeks thereafter in metastatic TNBC did not reveal unexpected toxicities more than anticipated (with Pembro alone) with the described combination. An approach of the disclosure is illustrated by FIG. 2.

Based in part on experience with vaccines against melanoma, colorectal cancer, glioma, and prostate cancer, and when given the benefit of the present disclosure, the feasibility and safety of the intralymphatic, intranodal, and intradermal routes of DC administration are encompassed by the present invention. Further, these approaches have demonstrated clinical responses and long-term disease stabilizations even in patients with particularly aggressive recurrent primary brain cancers (high-grade gliomas: AA and GBM). Thus, it is expected that the present disclosure, which differs from prior approaches in a number of ways, including but not limited to use of different combinations of peptides that have not previously been used in the described vaccines, will be safe and effective for use in breast cancer patients, including but not necessarily limited to breast cancer patients that have metastatic breast cancer that has manifest in the brain. The described approach is expected to therefore overcome the immunosuppressive tumor microenvironment in the brain. These clinical data enable analysis by manipulating the TME using systemic CKM, and analyzing increased accumulation of total (flow cytometry) and tumor-specific (ELISpot) CTLs and Th1 cells within the solid fraction of the (peripheral; biopsy-accessible) breast tumors, allowing the clinical benefit in patients with brain-metastatic parenchymal disease.

Since local expression of PD-1, PD-L1 and PD-L2 limits the scope of tumor-specific immunity, but simultaneously is a strong predictor of patients' clinical responses to PD-1 blockade, the present disclosure supports use of immunotherapies involving CKM-driven increases in intratumoral CTL and Th1 cell infiltration to convert “cold” checkpoint-resistant tumors, such as BMBC and other forms of breast cancer into checkpoint responsive ones.

The following description provides examples of preparation and use of compositions and methods of the disclosure, some of which are prophetic.

### Example 1

### Preparation of αDC1 Vaccines

Mononuclear cells are isolated from a leukaphereses product. The DC vaccine are prepared in a cGMP facility of the cGMP.

Cancer Vaccine and Dendritic Cell Therapies: TCPSR and the final product is vialed and cryopreserved. Vaccines, if shipped, can be frozen and thawed on the day of administration. Antigen-loaded DCs used in the vaccine are suspended in 5% human serum albumin (HSA) and delivered to the clinic for administration. Each syringe will be labeled with a custom-designed label, identifying the subject and the vaccine.

### Chemistry, Manufacturing and Control (CMC):

Harvesting of PBMC: Consented patients who are entered on protocol will undergo a 90-minute leukapheresis. The leukapheresis procedure involves the removal of blood from a vein in one arm, passage of the blood through a centrifuge to remove the white blood cells (WBC), and return of the remaining blood components (plasma, RBC, platelets) to the patient's vein in the other (or same) arm. No more than 15% of the patient's total blood volume is withdrawn at any one time as the blood is processed. Every attempt is made to use two peripheral IV lines for this procedure; though if that is not possible, a central line may be necessary. The subject must be cleared by physician to undergo the leukapheresis procedure and is routinely screened for blood pressure and vital signs prior to the procedure.

Processing of PBMC: After the leukapheresis collection, the product is hand-delivered to the ITC where processing for monocyte isolation occurs within 24 hr. Monocytes are isolated by density centrifugation. All processes and washing procedures are performed under sterile conditions. An aliquot of the monocyte fraction is analyzed by flow cytometry for purity by assessing the percentages of monocytes (CD14+) and lymphocytes (CD3+ T cells) and must have ≤30% lymphocyte contamination prior to cryopreservation. Prior to cryopreservation, an aliquot of the monocytes is tested for sterility. The records of results of sterility testing are maintained in the patient electronic batch record. Monocytes may be held for at 4° C. for <24 h if used for fresh DC culture. Additional monocytes may be cryopreserved. The labeled vials of monocytes are stored. Depending on the monocyte recovery from the leukapheresis product, multiple batches of DC vaccine may be made from the cryopreserved monocyte and each batch will be given a unique lot number.

DC culture: To generate a batch of DC vaccine, monocytes fresh or cryopreserved monocytes may be used. When using cryopreserved cells, the Patient's Name, Unique lab identifier, and specimen accession number on the label are verified by two lab members, and the cells are thawed. The final wash supernatant will be tested for bacterial sterility testing. Monocytes are resuspended at 1.2×10⁶ cells/mL in antibiotic-free RPMI medium, seeded to T25 flasks and incubated for 1 hr at 37° C. in 5% CO2 to allow monocytes to adhere to the plastic. The media and non-adherent cells are removed and the flasks are washed once with RPMI. The monocytes are cultured at 37° C. in 5% CO2 in antibiotic-free serum-free CellGenix DC medium containing 200 U/mL IL4 and 200 U/mL GMCSF for 6 days. On day 3±1, the cytokines in the cultures are replenished by removing 50% of the culture media and replacing with equal volume of CellGenix DC medium containing 400 U/mL IL4 and 400 U/mL GM-CSF. Each flask is labeled with the Patient Name, Patient Identification Number, and DC batch number.

DC maturation: On day 6 of monocyte culture, the DC are matured in presence of cytokines, TLR ligands, and Her2/3 class II peptides (see below) for 18±2 h. As with feeding, the maturation cytokines are added after 50% of the media has been removed. The cytokines used to generate αDC1 are GM-CSF, IFNα, IFNγ, IL1B, TNFα and poly-I:C.

HER-2/neu and HER-3 ECD and ICD peptides. Recombinant HER-2/neu peptides p42-56 (HLDMLRHLYQGCQVV) (SEQ ID NO:3), p98-114(RLRIVRGTQLFEDNYAL) (SEQ ID NO:4), p328-345(TQRCEKCSKPCARVCYGL) (SEQ ID NO:5), p776-790 (GVGSPYVSRLLGICL) (SEQ ID NO:6), p927-941 (PAREIPDLLEKGERL) (SEQ ID NO:7) and p1166-1180 (TLERPKTLSPGKNGV) (SEQ ID NO:8); HER-3 ECD peptides P81 (aa 401-415 SWPPHMHNFSVFSNL) (SEQ ID NO:9), P84 (aa 416-430 TTIGGRSLYNRGFSL) (SEQ ID NO:10), P91 (aa 451-465 AGRIYISANRQLCYH) (SEQ ID NO:11), HER-3 ICD peptides P38 (aa 850-864 VADFGVADLLPPDDK) (SEQ ID NO:12), P41 (aa 865-879 QLLYSEAKTPIKWMA) (SEQ ID NO:13), P52 (aa 920-934 VPDLLEKGERLAQPQ) (SEQ ID NO:14), P86 (aa 1090-1114 GCLASESSEGHVTGS) (SEQ ID NO:15) and P89 (aa 1115-1129 EAELQEKVSMCRSRS) (SEQ ID NO:16) have been made at BACHEM, Torrance, CA. These peptides will be >95% pure and certificate of analysis will be provided for each. The peptides will be stored lyophilized and reconstituted in sterile DMSO, diluted with sterile serum free media for use. All class II peptides will be added to DC cultures at the time of initiation of their maturation (day 6-7).

HER-2/neu MHC class I binding peptides. Three MHC class I binding peptides P369-377 (KIFGSLAFL) (SEQ ID NO:1) and P689-697 (RLLQETELV) (SEQ ID NO:2) have been synthesized at BACHEM, Torrance, CA. These peptides will be >95% pure and certificates of analysis will be provided for each peptide. The peptides will be stored lyophilized and reconstituted in sterile DMSO, diluted with sterile serum free media for use at the final stages of DC loading with antigens.

DC harvest, peptide loading and cryopreservation: To harvest mature αDC1, the flasks are placed 4° C. for 10-30 min for the semi-adherent cells to become less adherent. The cells are then harvested and each flask washed 2× with cold PBS. An aliquot of undiluted culture media is reserved after the cells are pelleted for mycoplasma and endotoxin testing. The cell pellets will be washed 2× in PBS. After the final wash, the cells will be resuspended in CellGenix DC media, counted, and assessed for viability using Trypan Blue exclusion assay. The cell concentration is adjusted to 1×10⁶ αDC1/mL for peptide loading. Her 2/3 class I peptides are loaded at a final concentration of 50 μg each peptide per 1×10⁶αDC1. The αDC1 and peptides are incubated for 3-4 h at 37° C., 5% CO2. The cells are then washed 2× in Cell Genix DC media. After the final wash, the cells will be resuspended in PBS, counted, and assessed for viability using Trypan Blue exclusion assay. An aliquot of DCs is removed for phenotyping and potency testing. A second aliquot of cells is added to the reserved culture media for mycoplasma and endotoxin testing.

The DCs are cryopreserved using in-house prepared freezing media (10% DMSO+40% humanAB serum in CellGenix DC medium). DCs are cryopreserved at 3-10×10⁶ cells/mL/vial, depending on batch recovery. The vials are labeled with the Patient Name, Patient Identification Number, DC batch number and Cells/vial. The cells are frozen in rate-controlled freezing containers and then transferred to long-term storage in liquid nitrogen vapor.

DC phenotype and purity: The DCs generated in culture are assessed for purity by differential cell count (DC vs lymphocytes) using a hemocytometer. This is confirmed by flow cytometric analysis. To determine purity, live cells are analyzed for expression of CD3 and CD14. To be released, the product must be >70% pure (i.e. not contain >30% CD3+ cells). To assess DC phenotype, aliquots of the final tumor-loaded αDC1 product are incubated with fluorochrome-conjugated antibodies to assess the expression of monocyte-derived DC markers [CCR7*, CD80*, CD86, HLA-DR and Isotype controls (* CCR7 and CD80 are followed for informational use only and are not part of release criteria)]. The phenotype of mature DCs is determined by gating on the live cell population. Release criteria is that >70% of the live CD45+ cells are HLA-DR+ and CD86+.

Potency (for correlative studies only, not a release criterion): IL-12p70 is a cytokine produced by DCs to promote the type-1 immune responses which are desirable for anti-tumor immune responses. This functional characteristic is used to assess potency of the DC for correlative studies but is not part of the release criteria. Upon harvest, an aliquot of DC is incubated with and without CD40L-expressing cells for 24 h at 37° C. The supernatants are harvested and analyzed for IL12p70 heterodimer by ELISA.

Components of the Chemokine-Modulating (CKM) Regimen and their Application

Celecoxib: A sulfa non-steroidal anti-inflammatory drug (NSAID) used in the treatment of osteoarthritis, rheumatoid arthritis, acute pain, painful menstruation and menstrual symptoms, and to reduce numbers of colon and rectum polyps in patients with familial adenomatous polyposis. Other Names: Celebrex, Celebra or Onsenal (commercially available). Formulation and packaging: Celecoxib as capsules in the following dosages: 100 mg and 200 mg. Drug Administration: 200 mg twice daily by mouth on days when the CKM regimen is administered.

Interferon Alpha-2b: A drug approved around the world for the treatment of chronic hepatitis C, chronic hepatitis B, hairy cell leukemia, chronic myelogenous leukemia, multiple myeloma, follicular lymphoma, carcinoid tumor, and malignant melanoma. Interferon alpha-2b has many drug classifications including anti-infective, anti-neoplastic, antiproliferative, antiviral and immunological agent. Formulation and Packaging: 50 million units/mL; lyophilized powder which must be reconstituted prior to administration. Vial size: 50 million units/vial. Diluent: Compatible with normal saline, Ringer's injection, lactated Ringer's, and 5% sodium bicarbonate injection. Interferon Alpha-2b should be reconstituted with 1 mL to reach a final concentration of 10:1. IV dose should be diluted in sodium chloride 0.9%/100 mL and given over 20 minutes. The final concentration of INTRON® A should not be less than 10 million IU/100 mL. Preparing and Dispensing: The lyophilized product is reconstituted as directed by the manufacturer. Interferon Alpha-2b will be prepared as per SOC for IV injection. Drug administration: Interferon Alpha-2b (20 million units/m²) will be administered intravenously over 20 minutes on days when the CKM regimen is administered (i.e., IV X 6 doses and per schedule). Powder for injection should be stored at 2 to 8° C. (36-46° F.). After reconstitution, the solution should be used immediately but may be stored up to 24 hours at 2-8° C. (36-46° F.).

Rintatolimod (poly IC analog). Other Names: PolyIC₁₂ U, Ampligen®, poly I: polyC₁₂ U; Polyinosinic: polycytidylic-polyuridylic acid; polyriboinosinic/polyribocytidylic (uridylic) acid. Formulation: Rintatolimod is supplied as a liquid solution in glass bottles containing 200 mg (100 mg in case of toxicity) per 80 mL. Rintatolimod is a colorless solution containing 2.5 mg/mL in physiological salts (0.15 M NaCl, 0.01 M phosphate, 0.001 M Mg++). The product does not contain preservatives or antioxidants. Drug Administration: Rintatolimod 200 mg will be administered by intravenous infusion after Interferon Alpha-2b on days of the CKM regimen. The initial administration begins at a slow rate of infusion (approximately 20 cc/hour) and increase to 40 cc/hour after 30 minutes. Tubing is flushed with 30 to 50 mL of normal saline solution upon completion.

### Example 2

This Example provides additional, non-limiting description on preparation and use of the described vaccines and agents.

As described above, the disclosure relates to a biologic product administered to patients in the form of dendritic cells, in combination with other agents. The biologic product is derived from the patient's own peripheral blood monocytes by culture in the presence of cytokines and thus, represents an autologous biologic product. The preparation of the cells does not involve any form of genetic manipulation.

TABLE 1
Example 2: Reagents Used in Manufacture and their Tabulation

| | | | |
|---|---|---|---|
| | | Stock | Final (culture) |
| Reagent | Manufacturer | concentration | concentration |

A. Media and General Reagents

| | | | |
|---|---|---|---|
| Ficoll-Paque Plus | GE healthcare Biosciences | N/A | N/A |
| Percoll | GE healthcare Biosciences | N/A | N/A |
| Phosphate-Buffered Saline | Mediatech, Inc. | N/A | N/A |
| 10× Acidic PBS | made in-house; see section | N/A | N/A |
| | 6.1 of the SOP PR.3200 | | |
| RPMI-1640 medium | Lonza | N/A | N/A |
| CellGenix DC media | CellGenix | N/A | N/A |
| 5% HSA | Pharmacy | N/A | N/A |
| DMSO | Protide Pharmaceuticals | N/A | N/A |
| Human AB serum | Gemini Bioproducts | N/A | N/A |

B. Growth Factors, Recombinant Cytokines and
Other DC Growth and Maturation Factors

| | | | |
|---|---|---|---|
| rhGM-CSF | Leukine, pharmaceutical | 2 × 10⁵ U/mL | 200 U/mL |
| | grade, known as purchased | | |
| | through the pharmacy | | |
| rhIL4 | Miltenyi | 2 × 10⁵ U/mL | 200 U/ml |
| rhIL1β | Miltenyi | 12.5 μg/mL | 12.5 ng/ml |
| rhTNFα | Miltenyi | 5 μg/mL | 5 ng/ml |
| rhIFNα | pharmaceutical grade, known | 1 × 10⁶ U/mL | 1000 U/mL |
| | as Intron A, purchased | | |
| | through the RPCI pharmacy | | |
| rhIFNγ | Miltenyi | 5 × 10⁵ IU/mL | 500 U/mL |
| poly I:C | Invivogen | 1 mg/mL | 10 mcg/mL |

C. Antigenic Peptides

| | | |
|---|---|---|
| HER2 MHC I-P369-377 | 1 mg/ml | 10 mcg/ml |
| KIFGSLAFL (SEQ ID NO: 1) | | |
| HER2 MHC I-P689-697 | 1 mg/ml | 10 mcg/ml |
| RLLQETELV (SEQ ID NO: 2) | | |
| HER2 MHC II-P42-56 | 1 mg/ml | 2 mcg/ml |
| HLDMLRHLYQGCQVV (SEQ ID NO: 3) | | |
| HER2 MHC II-P98-114 | 1 mg/ml | 2 mcg/ml |
| RLRIVRGTQLFEDNYAL (SEQ ID NO: 4) | | |
| HER2 MHC II-P328-345 | 1 mg/ml | 2 mcg/ml |
| TQRCEKCSKPCARVCYGL (SEQ ID NO: 5) | | |
| HER2 MHC II-P776-790 | 1 mg/ml | 2 mcg/ml |
| GVGSPYVSRLLGICL (SEQ ID NO: 6) | | |
| HER2 MHC II-P927-941 | 1 mg/ml | 2 mcg/ml |
| PAREIPDLLEKGERL (SEQ ID NO: 7) | | |
| HER2 MHC II-P1166-1180 | 1 mg/ml | 2 mcg/ml |
| TLERPKTLSPGKNGV (SEQ ID NO: 8) | | |
| HER3 MHC II ECD-P81 (aa 401-415) | 1 mg/ml | 2 mcg/ml |
| SWPPHMHNFSVFSNL (SEQ ID NO: 9) | | |
| HER3 MHC II ECD-P84 (aa 416-430) | 1 mg/ml | 2 mcg/ml |
| TTIGGRSLYNRGFSL (SEQ ID NO: 10) | | |
| HER3 MHC II ECD-P91 (aa 451-465) | 1 mg/ml | 2 mcg/ml |
| AGRIYISANRQLCYH (SEQ ID NO: 11) | | |
| HER3 MHC II ICD-P38 (aa 850-864) | 1 mg/ml | 2 mcg/ml |
| VADFGVADLLPPDDK (SEQ ID NO: 12) | | |
| HER3 MHC II ICD-P41 (aa 865-879) | 1 mg/ml | 2 mcg/ml |
| QLLYSEAKTPIKWMA (SEQ ID NO: 13) | | |
| HER3 MHC II ICD-P52 (aa 920-934) | 1 mg/ml | 2 mcg/ml |
| VPDLLEKGERLAQPQ (SEQ ID NO: 14) | | |
| HER3 MHC II ICD-P86 (aa 1090-1114) | 1 mg/ml | 2 mcg/ml |
| GCLASESSEGHVTGS (SEQ ID NO: 15) | | |
| HER3 MHC II ICD-P89 (aa 1115-1129) | 1 mg/ml | 2 mcg/ml |
| EAELQEKVSMCRSRS (SEQ ID NO: 16) | | |

Since the biologic activity potency (such as specific activity) of the cytokines and other factors used to grow and mature DCs is established by their manufacturers in bio-assays which involve other than DCs cell types, the present disclosure provides for up to two-fold adjustment of their concentrations (down to 50% or up to 200% of the concentrations listed above). Such decisions can be made after validation of the need to adjust the concentrations in at least 3 separate tests (compared to 2 tests needed for straight validation of the new reagent to be used at the same concentration).

In embodiments, DCs are prepared in antibiotic-free serum-free CellGenix DC medium. The disclosure includes use of serum-free media; such as X-Vivo or AIM-V.

In an embodiment, the active drug component is CD14+DCs which will comprise of >70%. The drug product will also contain CD3+ cells which will be <30%.

### Method of Cell Collection/Processing/Culture Conditions

All of the steps in the process of DC generation can be performed under aseptic conditions in laminar flow units, using single-use sterile pipets for cell feedings, transfer, and sampling. The antigenic peptides may be tested for stability.

Harvesting of PBMC: Patients undergo a 90-minute leukapheresis. The leukapheresis procedure involves the removal of blood from a vein in one arm, passage of the blood through a centrifuge to remove the white blood cells (WBC), and return of the remaining blood components (plasma, RBC, platelets) to the patient's vein in the other (or same) arm. No more than 15% of the patient's total blood volume is withdrawn at any one time as the blood is processed. Every attempt is made to use two peripheral IV lines for this procedure; though if that is not possible, a central line may be necessary.

Processing of PBMC: After the leukapheresis collection, the apheresis product is processed for monocyte isolation within 24 hr. Monocytes are isolated by density centrifugation in a two day process. All processes and washing procedures are performed under sterile conditions. An aliquot of the monocyte fraction is analyzed by flow cytometry for purity by assessing the percentages of monocytes (CD14+) and lymphocytes (CD3+ T cells) and may have ≤30% lymphocyte contamination prior to cryopreservation or proceed to the next step. Prior to cryopreservation, an aliquot of the monocytes is tested for sterility.

Monocytes may be held for at 4° C. for <24 h if used for fresh DC culture. Additional monocytes will be cryopreserved. The labeled vials of monocytes are stored in the vapor phase of liquid nitrogen until vaccine preparation. Depending on the monocyte recovery from the leukapheresis, multiple batches of DC vaccine may be made from the cryopreserved monocyte and each batch will be given a unique lot number.

### DC Culture, Maturation and Class II Peptide Loading

DC culture: To generate a batch of DC vaccine, fresh monocytes or cryopreserved monocytes may be used. The final wash supernatant will be tested for bacterial sterility testing. Monocytes are resuspended at 1.2×10⁶ cells/mL in antibiotic free RPMI medium, seeded to T25 flasks and incubated for 1 hr at 37° C. in 5% CO2 to allow monocytes to adhere to the plastic. The media and non-adherent cells are removed, and the flasks are washed once with RPMI. The monocytes are cultured at 37° C. in 5% CO2 in antibiotic-free serum-free CellGenix DC medium (or analogous serum-free medium; such as X-Vivo or AIM-V) containing 200 U/mL IL4 and 200 U/mL GM-CSF (since the potency of these cytokines is established by manufacturers in other assays; the concentrations of both cytokines can be increased up to 2-fold, to accommodate batch-to batch difference) for 6 days. On day 3+1, the cytokines in the cultures are replenished by removing 50% of the culture media and replacing with equal volume of CellGenix DC medium containing 400 U/mL IL4 and 400 U/mL GM-CSF.

DC maturation and MHC class II peptide loading: On day 6 of monocyte culture, the DC are matured in presence of cytokines and TLR ligands, in the presence of class II-restricted Her2/Her3 peptides (2 μg each; see Table 1) for 18±2 h as per SOP PR 3600. As with feeding, the maturation cytokines are added after 50% of the media has been removed. The final concentration of the cytokines used to generate αDC1 is 200 U/mL GM-CSF, 1000 U/mL IFNα, 500 U/mL IFNγ, 12.5 ng/ML IL1β, 5 ng/ml TNFα and 10 μg/mL poly-I:C. Suitable modifications of the described concentrations will be apparent to those skilled in the art based on the present disclosure, and such concentration modifications are included within the scope of the invention.

### Class I Ag Loading and Final Harvest

DC harvest, peptide loading and cryopreservation: Mature αDC1 are harvested. The flasks are placed 4° C. for 10-30 min for the semi-adherent cells to become less adherent. The cells are then harvested and each flask is washed 2× with cold PBS. An aliquot of undiluted culture media is reserved after the cells are pelleted for mycoplasma and endotoxin testing. The cell pellets will be washed 2× in PBS. After the final wash, the cells will be resuspended in CellGenix DC media, counted, and assessed for viability using Trypan Blue exclusion assay. The cell concentration is adjusted to 1×10⁶ αDC1/mL for peptide loading. Both HER2 class I peptides are loaded at a final concentration of 10 μg each class I Her 2 peptide per 1×10⁶ αDC1. The αDC1 and peptides are incubated for 3-4 h at 37° C., 5% CO2. The cells are then washed 2×in PBS. After the final wash, the cells will be resuspended in PBS, counted, and assessed for viability using Trypan Blue exclusion assay. An aliquot of DCs is removed for phenotyping and potency testing. A second aliquot of cells is added to the reserved culture media for mycoplasma and endotoxin testing as well as for sterility test and Gram staining.

### Timing and Intermediate Storage

The DCs are cryopreserved as per SOP PR 3300 using in-house prepared freezing media (10% DMSO+40% human AB serum in CellGenix DC medium). DCs are cryopreserved at 5-30×10⁶ cells/mL/vial, depending on batch recovery. The cells are frozen in rate-controlled freezing containers and then transferred to long-term storage in liquid nitrogen vapor.

### Final Formulation

Vaccine preparation and delivery: On the day of vaccine administration, a vial of DC vaccine is removed from cryopreservation and prepared for administration and the cells are quickly thawed in a 37° C. water bath. The thawed DC are washed twice in clinical grade 5% human serum albumin (HSA), assessed for viability by Trypan Blue exclusion and counted. One 1 mL syringe will be loaded with 10×10⁶ DC resuspended in 0.2 mL 5% HSA. The syringe is stored at 4° C. for up to 6 h until the cells are administered as determined by the clinical protocol.

### Example 3

This Example provides a description that relates to use of intact polypeptides that are internally processed by DCs for use in prophylaxis or therapy of cancer as described above.

In this Example, DCs loaded with an intact Her3 protein are provided to thereby include DCs that present a wide range of antigenic epitopes, including the previously-identified promiscuous MHC class II binding peptides: HER-3 extracellular domain (ECD) peptides:

| | P81 |
|---|---|
| (SEQ ID NO: 9) | |
| | (aa 401-415 SWPPHMHNFSVFSNL), |
| | P84 |
| (SEQ ID NO: 10) | |
| | (aa 416-430 TTIGGRSLYNRGFSL), |
| | and |
| | P91 |
| (SEQ ID NO: 11) | |
| | (aa 451-465 AGRIYISANRQLCYH), |

HER-3 intracellular domain (ICD) peptides:

| | P38 |
|---|---|
| (SEQ ID NO: 12) | |
| | (aa 850-864 VADFGVADLLPPDDK), |
| | P41 |
| (SEQ ID NO: 13) | |
| | (aa 865-879 QLLYSEAKTPIKWMA), |
| | P52 |
| (SEQ ID NO: 14) | |
| | (aa 920-934 VPDLLEKGERLAQPQ), |
| | P86 |
| (SEQ ID NO: 15) | |
| | (aa 1090-1114 GCLASESSEGHVTGS), |
| | P89 |
| (SEQ ID NO: 16) | |
| | (aa 1115-1129 EAELQEKVSMCRSRS). |

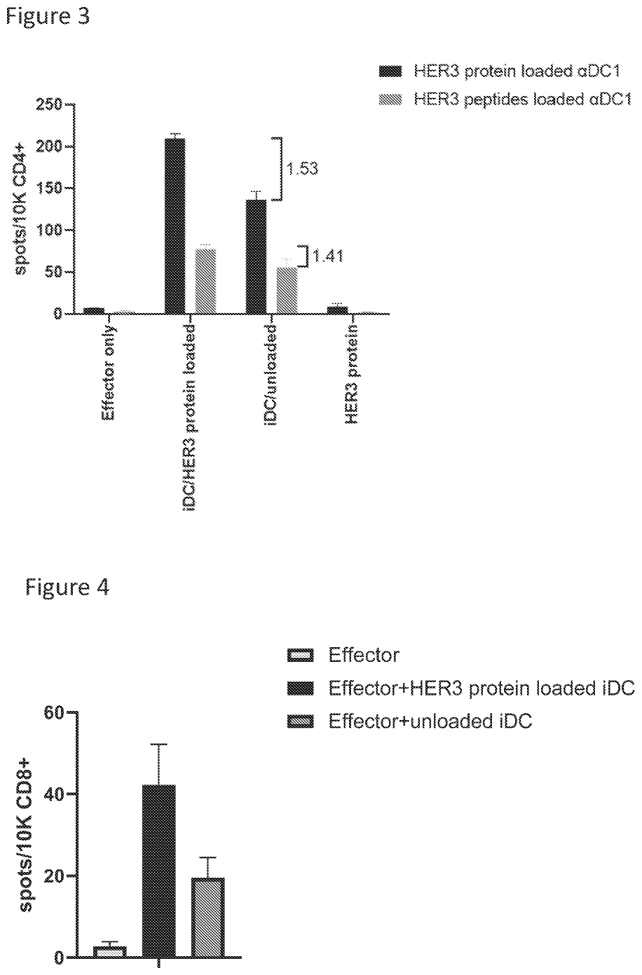

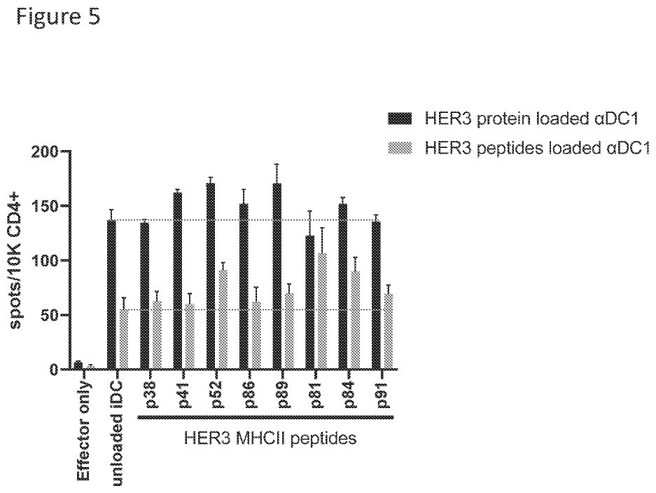

Results related to this Example are presented in FIGS. 3, 4, and 5. It will be recognized that this Example illustrates certain advantages using intact protein over the use of αDC1s (e.g., immature DCs) loaded with the Her3 synthetic peptides. Without intending to be limited by any particular theory, it is considered that some of the advantages include the observations that αDC1s loaded with Her3 protein are similarly or more immunogenic that αDC1s loaded with the previously defined Her3 class II restricted peptides in inducing CD4+ T cell responses (FIG. 3). nexpectedly, αDC1s loaded with Her3 protein may also induce Her3-specific responses within CD8 population (FIG. 4). αDC1s loaded with Her3 protein are expected to induce a wider spectrum of Her3-specific responses within CD4+ and CD8+ T cell populations (FIG. 5). αDC1s loaded with Her3 protein may induce activation of CD4+ and CD8+ T cell populations, responding to additional epitopes present in DCs (FIGS. 3-5). These properties may enhance the ability to activate high avidity T cells that are able to recognize weak antigens present on human DCs. Additionally, proteins and protein-derived peptides may persist on the antigen-loaded DCs longer than peptides, which could benefit vaccines' performance in vivo.

1. A method for treating cancer in an individual in need thereof, the cancer comprising at least one tumor, the method comprising administering to the individual:
a) a combination of autologous dendritic cells i) loaded in vitro with at least one major histocompatibility complex (MHC) Class II- and/or at least one MHC class I-restricted Her2 or Her3 peptide, or ii) exposed in vitro to an intact Her3 protein to process and display Her3 peptides, or a combination or ii) and iii); and optionally,
b) a combination of agents, said combination of agents having a tumor selective chemokine-modulating (CKM) effect.

2. The method of claim 1, wherein the dendritic cells are alpha-type-1 dendritic cells (αDC1s), matured in interferon alpha (IFNα) and interferon gamma (IFNγ), and optionally in the presence of one or a combination of interleukin 1 (IL-1), tumor necrosis factor (TNF), and poly-I:C.

3. The method of claim 1, wherein the dendritic are matured in the presence of the combination of IFNα and IFNγ, and in the presence of at least two of the IL-1, TNF, and poly-I:C.

4. The method of claim 1, wherein the individual has been diagnosed with Brain Metastatic Breast Cancer (BMBC).

5. The method of claim 1, wherein the at least one MHC Class II- and/or the at least one MHC class I-restricted Her2 or Her3 peptides comprise at least one of:

| | HER2 MHC I - P369-377 |
|---|---|
| (SEQ ID NO: 1) | |
| | KIFGSLAFL |
| | HER2 MHC I - P689-697 |
| (SEQ ID NO: 2) | |
| | RLLQETELV |
| | HER2 MHC II - P42-56 |
| SEQ ID NO: 3) | |
| | HLDMLRHLYQGCQVV |
| | HER2 MHC II - P98-114 |
| (SEQ ID NO: 4) | |
| | RLRIVRGTQLFEDNYAL |
| | HER2 MHC II - P328-345 |
| (SEQ ID NO: 5) | |
| | TQRCEKCSKPCARVCYGL |
| | HER2 MHC II - P776-790 |
| (SEQ ID NO: 6) | |
| | GVGSPYVSRLLGICL |
| | HER2 MHC II - P927-941 |
| (SEQ ID NO: 7) | |
| | PAREIPDLLEKGERL |
| | HER2 MHC II - P1166-1180 |
| (SEQ ID NO: 8) | |
| | TLERPKTLSPGKNGV |
| | HER3 MHC II ECD - P81(aa 401-415) |
| (SEQ ID NO: 9) | |
| | SWPPHMHNFSVFSNL |
| | HER3 MHC II ECD - P84(aa 416-430) |
| (SEQ ID NO: 10) | |
| | TTIGGRSLYNRGFSL |
| | HER3 MHC II ECD - P91(aa 451-465) |
| (SEQ ID NO: 11) | |
| | AGRIYISANRQLCYH |
| | HER3 MHC II ICD - P38(aa 850-864) |
| (SEQ ID NO: 12) | |
| | VADFGVADLLPPDDK |
| | HER3 MHC II ICD - P41(aa 865-879) |
| (SEQ ID NO: 13) | |
| | QLLYSEAKTPIKWMA |
| | HER3 MHC II ICD - P52(aa 920-934) |
| (SEQ ID NO: 14) | |
| | VPDLLEKGERLAQPQ |
| | HER3 MHC II ICD - P86(aa 1090-1114) |
| (SEQ ID NO: 15) | |
| | GCLASESSEGHVTGS |
| | HER3 MHC II ICD - P89(aa 1115-1129) |
| (SEQ ID NO: 16) | |
| | EAELQEKVSMCRSRS |

6. The method of claim 5, wherein the at least one Class II peptide is selected from:
HER-3 extracellular domain (ECD) peptides:
P81 (aa 401-415 SWPPHMHNFSVFSNL) (SEQ ID NO:9),
P84 (aa 416-430 TTIGGRSLYNRGFSL) (SEQ ID NO:10), and
and P91 (aa 451-465 AGRIYISANRQLCYH) (SEQ ID NO:11),
HER-3 intracellular domain (ICD) peptides:
P38 (aa 850-864 VADFGVADLLPPDDK) (SEQ ID NO:12),
P41 (aa 865-879 QLLYSEAKTPIKWMA) (SEQ ID NO:13),
P52 (aa 920-934 VPDLLEKGERLAQPQ) (SEQ ID NO:14),
P86 (aa 1090-1114 GCLASESSEGHVTGS) (SEQ ID NO:15), and
P89 (aa 1115-1129 EAELQEKVSMCRSRS) (SEQ ID NO:16);
and wherein the at least one Class II peptide is selected from:
HER-2/neu peptide P369-377 (KIFGSLAFL) (SEQ ID NO:1) and P689-697 (RLLQETELV) (SEQ ID NO:2).

7. The method of claim 6, wherein a combination of at least two of the peptides are loaded onto the autologous dendritic cells.

8. The method of claim 6, wherein the combination of the at least two peptides comprises at least one Class II peptide, the combination optionally comprising at least one Class I peptide.

9. The method of claim 8, wherein the combination of the at least two peptides comprises a combination of at least one Class II peptide and at least one Class I peptide.

10. The method of claim 1, wherein the combination of agents having the tumor selective CKM effect comprises a combination selected from the group of agents consisting of:
i) a COX-2 inhibitor that is optionally Celecoxib.
ii) an interferon that is optionally human recombinant Interferon Alpha-2b, and
iii) a poly IC analog (double-stranded RNA) that is optionally Rintatolimod.

11. The method of claim 10, wherein the COX-2 inhibitor is the Celecoxib, the interferon is the human recombinant Interferon Alpha-2b, and the poly IC analog is optionally the Rintatolimod.

12. The method of claim 11, wherein the autologous dendritic cells comprise alpha dendritic cells that are matured in the presence of at least one of the peptides, and in the presence of cytokines, the cytokines comprising a combination of at least two of GM-CSF, IFNα, IFNγ, IL1β, TNFα and poly-I:C.

13. The method of claim 1, wherein approximately 10×10⁶ of peptide loaded alpha dendritic cells are administered to the individual, and wherein the alpha dendritic cells were optionally cryopreserved prior to said administering.

14. The method of claim 1, where administering the autologous dendritic cells is provided as a combination therapy with at least one of: an adjuvant, a cytokine, an inhibitor of at least one checkpoint molecule, or a suppressive factor.

15. The method of claim 14, wherein the combination therapy comprises the inhibitor of the checkpoint molecule.

16. The method of claim 15, wherein the autologous dendritic cells and the combination of CKM agents sensitizes the individual to the inhibitor of the checkpoint molecule.

17. The method of claim 16, wherein the inhibitor of the checkpoint molecule inhibits at least one of PD1, PD-L1 or PD-L2, or CTLA4.

18. The method of claim 17, wherein the inhibitor of the checkpoint molecule comprises Pembrolizumab.

19. A population of isolated alpha dendritic cells having loaded thereon one or more peptides of claim 6.

20. A population of isolated alpha dendritic cells having loaded thereon one or more peptides of claim 6, wherein the alpha dendritic cells are derived from a monocyte culture that is exposed to one or more peptides of claim 6.

21. A pharmaceutical composition comprising a population of isolated alpha dendritic cells as in claim 19.

22. A method comprising selecting an individual for treatment of a cancer, the cancer comprising at least one tumor, isolating a liquid biological sample comprising at least peripheral blood mononuclear cells (PBMCs) from the individual, separating monocytes from the PBMCs, culturing the monocytes in the presence of at least one peptide of claim 6 or an intact Her3 protein or a combination thereof, and optionally a combination of cytokines selected from GM-CSF, IFNα, IFNγ, IL1β, TNFα and poly-I:C, to thereby produce autologous alpha dendritic cells comprising MHC II or MHC I molecules, or a combination thereof, loaded with at least one of said peptides.

23. The method of claim 22, further comprising administering the alpha-type-1-polarized dendritic cells to the individual.

24. The method of claim 23, wherein the individual has been diagnosed with Brain Metastatic Breast Cancer (BMBC).

25. The method of claim 22, further comprising administering to the individual a combination of agents selected from the group of agents consisting of:
i) a COX-2 inhibitor that is optionally Celecoxib.
ii) an interferon that is optionally human recombinant Interferon Alpha-2b, and
iii) a poly IC analog that is optionally Rintatolimod.

26. The method of claim 25, further comprising administering to the individual least one of: an adjuvant, a cytokine, an inhibitor of at least one checkpoint molecule, or a suppressive factor.

27. The method of claim 26, wherein the combination therapy comprises the inhibitor of the checkpoint molecule.

28. The method of claim 27, wherein the autologous dendritic cells and the combination of CKM agents sensitize the individual to the inhibitor of the checkpoint molecule.

29. The method of claim 28, wherein the inhibitor of the checkpoint molecule inhibits at least one of PD1, PD-L1 or PD-L2, or CTLA4.

30. The method of claim 29, wherein the inhibitor of the checkpoint molecule comprises Pembrolizumab.

31. The method of claim 30, wherein the tumor was resistant to the inhibitor of the checkpoint molecule prior to the administration of the autologous alpha dendritic cells and the combination of CKM agents, and wherein said administration sensitizes the individual to the inhibitor of the checkpoint molecule.
